(19) United States
(12) Patent Application Publication
(10) Pub. No.: US 2023/0201342 A1
(43) Pub. Date: Jun. 29, 2023

(54) USE OF LENVATINIB PLUS ANTI-PD-1 MONOCLONAL ANTIBODY IN PREPARATION OF ANTI-HEPATOMA DRUG

(71) Applicant: Guangzhou X Cell Therapy Biomedical Tech Co., Ltd, Guangzhou (CN)

(72) Inventors: Tong Xiang, Guangzhou City (CN); Jun Wang, Guangzhou City (CN)

(73) Assignee: Guangzhou X Cell Therapy Biomedical Tech Co., Ltd, Guangzhou (CN)

(21) Appl. No.: 17/936,398

(22) Filed: Sep. 29, 2022

(30) Foreign Application Priority Data
Dec. 23, 2021 (CN) 202111590199.4

(51) Int. Cl. A61K 39/395; A61K 31/47; C07K 16/28; A61P 35/00

(52) U.S. Cl. CPC A61K 39/3955; A61K 31/47; C07K 16/2818; A61P 35/00; A61K 2039/545

(57) ABSTRACT

Some embodiments of the disclosure provide use of lenvatinib plus anti-PD-1 monoclonal antibody in preparation of an anti-hepatoma drug and belong to the technical field of medicine. In some embodiments, administration of the lenvatinib plus the anti-PD-1 monoclonal antibody promotes vascular “normalization” of hepatoma, while enhancing the infiltration of T lymphocytes in tumor tissue, thus significantly enhancing the therapeutic effect of hepatoma. In some embodiments, such administration may be used for the prophylaxis and treatment of the hepatoma.

### CROSS REFERENCE TO RELATED APPLICATION

This patent application claims priority to Chinese Patent Application No. 202111590199.4, filed on Dec. 23, 2021, the disclosure of which is incorporated by reference herein in its entirety.

### FIELD OF THE DISCLOSURE

The disclosure relates generally to the field of medicine. More specifically, the disclosure relates to use of lenvatinib plus anti-PD-1 monoclonal antibody in preparation of an anti-hepatoma drug.

### BACKGROUND

According to the World Cancer Report 2020 published by the WHO, primary hepatocellular carcinoma (hereinafter referred to as “hepatoma”) is a malignant tumor that ranks sixth in global cancer morbidity and third in mortality. However, in China, both of the morbidity and mortality of hepatoma account for nearly 50% of those all over the world, and China has been a country with the highest morbidity and mortality of hepatoma worldwide. A plurality of patients with hepatoma have lost opportunities of topical treatment such as radical treatment (for example, surgery, liver transplantation, or radiofrequency ablation) and chemotherapy embolization when seeing a doctor; plus, hepatoma has low sensitivity for systematic chemotherapy, resulting in poor outcomes of these patients with hepatoma. Therefore, there is an urgent need to find a new method for effectively treating advanced hepatoma.

In recent years, immunotherapy becomes an emerging therapy for tumors, where anti-PD-1 monoclonal antibody obtains an excellent efficacy in the treatment of a plurality of tumor types such as pulmonary carcinoma, melanoma, renal carcinoma, and head and neck carcinoma. In immunotherapy for advanced hepatoma, the anti-PD-1 monoclonal antibody is recommended as a second-line treatment for advanced hepatocellular carcinoma (HCC) recommended by Class I experts in 2020 edition of the CSCO Primary Liver Cancer Diagnosis and Treatment Guide. However, in practical clinic application, only about 20% of patients with hepatoma are benefited from the anti-PD-1 monoclonal antibody therapy. Therefore, the efficacy of the anti-PD-1 monoclonal antibody is limited, and there is an urgent need to explore more treatment strategies for improving the efficacy of anti-PD-1 monoclonal antibody immunotherapy in hepatoma.

Exertion of the efficacy of the anti-PD-1 monoclonal antibody depends on the infiltration of sufficient T cells in tumor tissue, and the key pathway of the infiltration of T cells into the tumor tissue is tumor-associated blood vessels. However, a large number of abnormal blood vessels are present in hepatoma tissues, which cause poor blood perfusion of tumor tissues, restrict the infiltration of T cells into the tumor tissue, and thus resist the exertion of the efficacy of the anti-PD-1 monoclonal antibody. Therefore, restoring the infiltration of T cells into the tumor tissue by vascular “normalization” in hepatoma tissues is a key to improve the efficacy of the anti-PD-1 monoclonal antibody.

At present, it has been found by gene sequencing that the regulation of abnormal vessel-associated genes in hepatoma tissues and the further research and development of reversion of abnormal blood vessels into “normalized” blood vessels is a very time-consuming process. If the reversion phenomenon of abnormal blood vessels of tumors into normal blood vessels is found from the existing drugs for the treatment of hepatoma, the exertion of the efficacy of the anti-PD-1 monoclonal antibody will be further promoted; more importantly, it substantially reduces the cost for the research and development of vascular “normalization” of hepatoma.

### SUMMARY

The following presents a simplified summary of the invention in order to provide a basic understanding of some aspects of the invention. This summary is not an extensive overview of the invention. It is not intended to identify critical elements or to delineate the scope of the invention. Its sole purpose is to present some concepts of the invention in a simplified form as a prelude to the more detailed description that is presented elsewhere.

In view of this, a first objective of the present disclosure is to provide use of lenvatinib plus anti-PD-1 monoclonal antibody in preparation of an anti-hepatoma drug.

Optionally, the present disclosure provides use of low-dose lenvatinib as a synergist for treating hepatoma with the anti-PD-1 monoclonal antibody.

Optionally, the anti-hepatoma drug may be a drug for inhibiting growth of hepatoma.

Optionally, the hepatoma may be primary hepatocellular carcinoma.

A second objective of the present disclosure is to provide an anti-hepatoma drug, where the drug includes lenvatinib and anti-PD-1 monoclonal antibody.

Optionally, a dose of the lenvatinib may be 10 mg/kg body weight/day.

Optionally, a dose of the anti-PD-1 monoclonal antibody may be 200 μg/kg body weight/3 days.

Optionally, the lenvatinib may be an oral drug, and the anti-PD-1 monoclonal antibody may be an intravenous drug.

Optionally, the drug may further include pharmaceutically acceptable excipients or carriers.

Early in 2018, lenvatinib had been approved as a first-line therapeutic drug for advanced hepatoma. The present disclosure finds that administration of low-dose lenvatinib enables vascular “normalization” of hepatoma, and may promote the infiltration of T cells into the tumor tissue. Administration of low-dose lenvatinib plus anti-PD-1 monoclonal antibody has more significant inhibitory effect on the growth of hepatoma than administration of high-dose lenvatinib plus anti-PD-1 monoclonal antibody. Thus, administration of low-dose lenvatinib plus anti-PD-1 monoclonal antibody may be used for the prophylaxis and treatment of the hepatoma.

### BRIEF DESCRIPTION OF THE DRAWINGS

Illustrative embodiments of the present disclosure are described in detail below with reference to the figures.

FIG. 1 illustrates effects of three different doses of lenvatinib on the size of hepatoma xenograft in the tumor tissue according to Example 1 of the present disclosure.

FIG. 2 illustrates effects of three different doses of lenvatinib on the percentage of CD8-positive T cells in the tumor tissue according to Example 1 of the present disclosure.

FIG. 3 illustrates effects of different doses of lenvatinib and expression of CD31, NG2, and CD8 in tumor tissue according to Example 1 of the present disclosure.

FIG. 4 illustrates results of CD31-positive volume per visual filed versus control group and various dose groups of lenvatinib according to Example 1 of the present disclosure.

FIG. 5 illustrates results of NG2-positive volume per visual filed versus control group and various dose groups of lenvatinib according to Example 1 of the present disclosure.

FIG. 6 illustrates results of relative value of NG2/CD31-positive volume versus control group and various dose groups of lenvatinib according to Example 1 of the present disclosure.

FIG. 7 illustrates results of relative value of CD8-positive cell count per visual field versus control group and various dose groups of lenvatinib according to Example 1 of the present disclosure.

FIG. 8 illustrates effects of administration of different doses of lenvatinib plus anti-PD-1 monoclonal antibody on the growth of hepatoma xenograft according to Example 2 of the present disclosure.

### DETAILED DESCRIPTION

The following describes some non-limiting embodiments of the invention with reference to the accompanying drawings. The described embodiments are merely a part rather than all of the embodiments of the invention. All other embodiments obtained by a person of ordinary skill in the art based on the embodiments of the disclosure shall fall within the scope of the disclosure.

In order to make the objectives, technical solutions and advantages of the present disclosure clearer, the technical solutions of the present disclosure will be described clearly and completely with reference to specific examples of the present disclosure. Apparently, the described examples are only a part of, not all of, the examples of the present disclosure. All other examples obtained by a person of ordinary skill in the art based on the examples of the present disclosure without creative efforts should fall within the protection scope of the present disclosure.

The experimental methods described in the following examples are conventional methods unless otherwise specified; the raw materials and additives may be obtained from conventional commercial sources unless otherwise specified.

### Example 1

Exploration of the optimal dose of lenvatinib for improving vascular normalization of mouse hepatoma xenograft.

1. Experimental Materials

(1) Drug: lenvatinib, chemical formula: C₂₁H₁₉ClN₄O₄, CAS NO: 417716-92-8;

(2) Cancer cells: mouse hepatoma cells (Hepa1-6); and

(3) Commercially available immunocompetent C57B/L mice.

2. Experimental Grouping

(1) Control group: blank control, namely hepatoma-bearing mice untreated with any drug;

(2) 3 mg/kg/day lenvatinib group: hepatoma-bearing mice treated with 3 mg/kg/day lenvatinib;

(3) 10 mg/kg/day lenvatinib group: hepatoma-bearing mice treated with 10 mg/kg/day lenvatinib; and

(4) 30 mg/kg/day lenvatinib group: hepatoma-bearing mice treated with 30 mg/kg/day lenvatinib.

3. Detection of the effects of different doses of lenvatinib on hepatoma xenograft by subcutaneous tumorigenicity assay of immunocompetent C57B/L mice.

(1) Separately, 1×10⁶ mouse hepatoma cells (Hepa1-6) were implanted into the subcutaneous stratum of the armpits of 20 NOD/SCID mice aged 3-4 weeks. The mice were randomized into four groups: a blank control group, a 3 mg/kg/day lenvatinib group, a 10 mg/kg/day lenvatinib group, and a 30 mg/kg/day lenvatinib group.

(2) When the size of the subcutaneous tumor reached 100-200 mm³: each mouse of the 3 mg/kg/day lenvatinib group was orally administered with 3 mg/kg/day lenvatinib by gastric gavage once a day; each mouse of the 10 mg/kg/day lenvatinib group was orally administered with 10 mg/kg/day lenvatinib by gastric gavage once a day; and each mouse of the 30 mg/kg/day lenvatinib group was orally administered with 30 mg/kg/day lenvatinib by gastric gavage once a day. According to the existing literature, 30 mg/kg was a routine dose of lenvatinib (Selleck) for laboratory mice.

(3) The tumor size was measured every two days, and the difference in tumor size was compared among groups.

(4) After 3 weeks, the mice were sacrificed, tumor tissues were resected, a part of fresh tumor tissue was separated and ground into a single cell suspension, and the proportion of positive cells labeled with CD45 and CD8 was detected by flow cytometry; meanwhile, the other part of tumor tissue was fixed and embedded, and vascular markers (CD31, α-SMA, and VEGFR2) and CD8-positive T cell infiltration were detected by immunohistochemistry.

4. Experimental Results

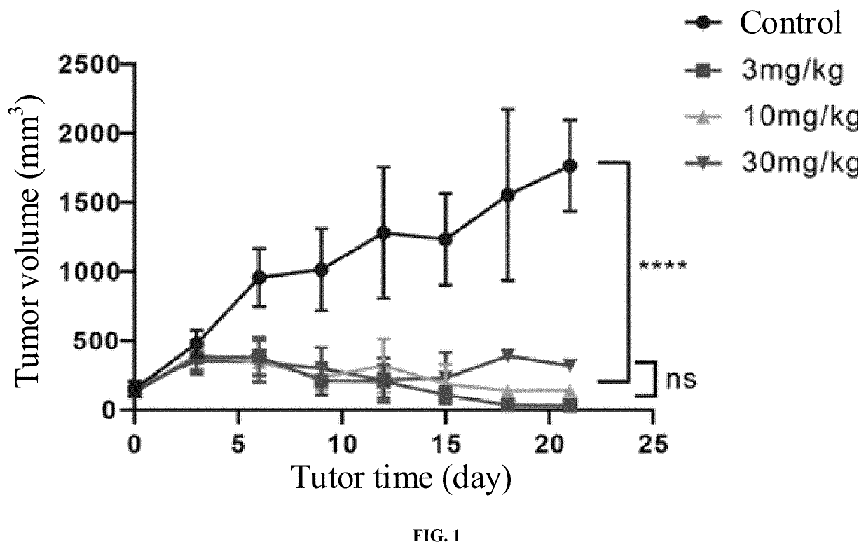

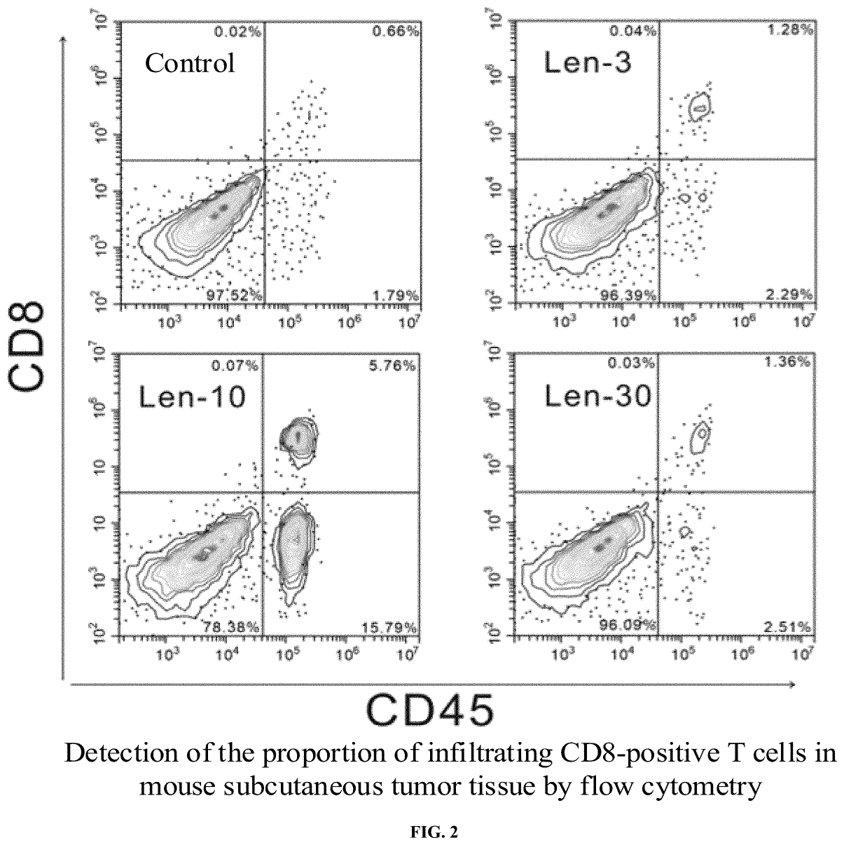

The results are shown in FIGS. 1 and 2. All of the three dose gradients of lenvatinib may inhibit the growth of hepatoma xenograft, and there is no significant difference in tumor growth among three groups of mice. Flow cytometry results suggest that the percentage of CD8-positive T cells in the tumor tissue of the 10 mg/kg/day dose group is significantly increased.

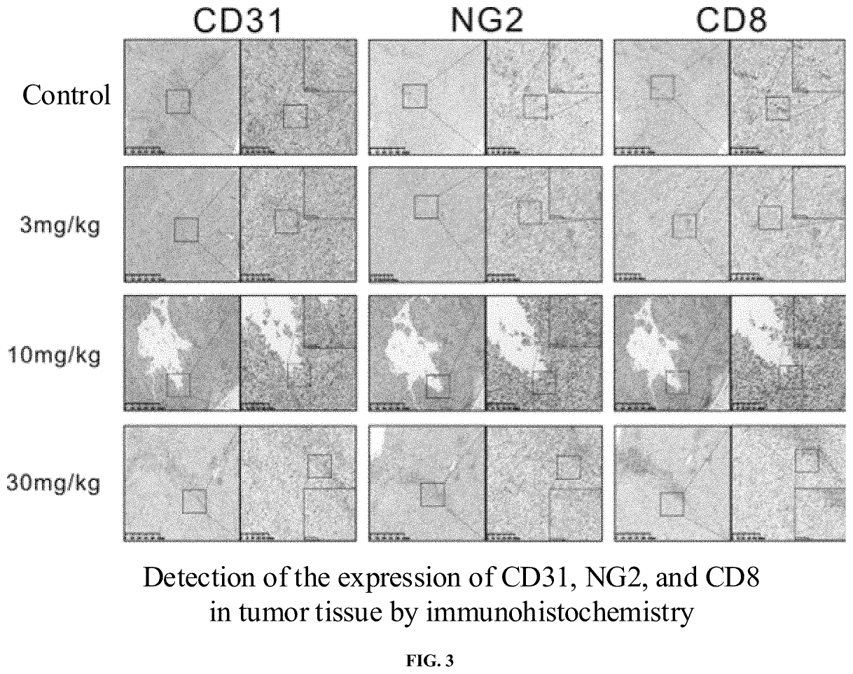

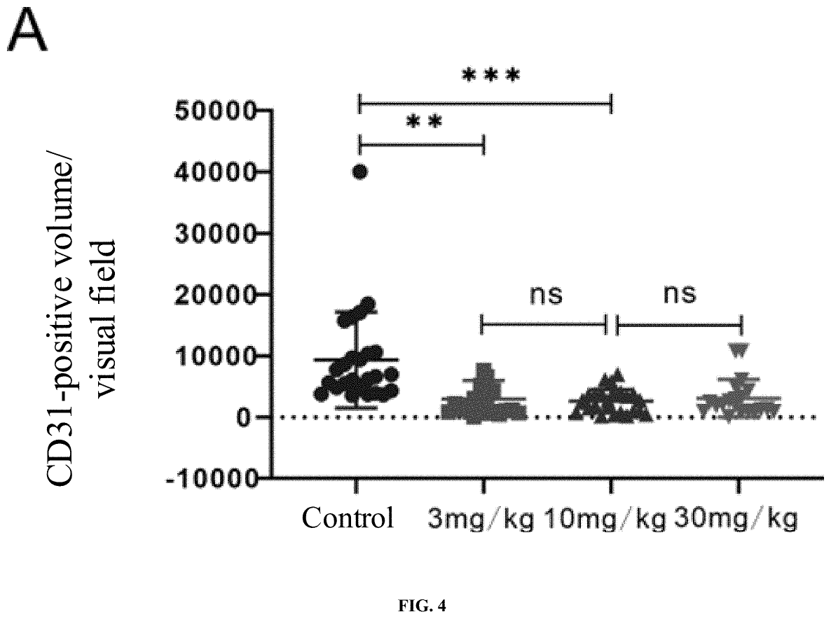

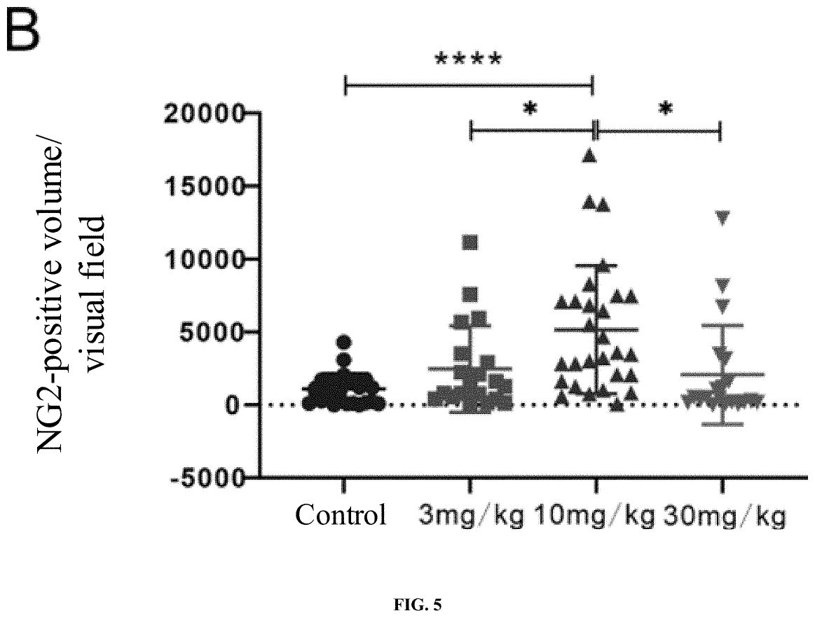

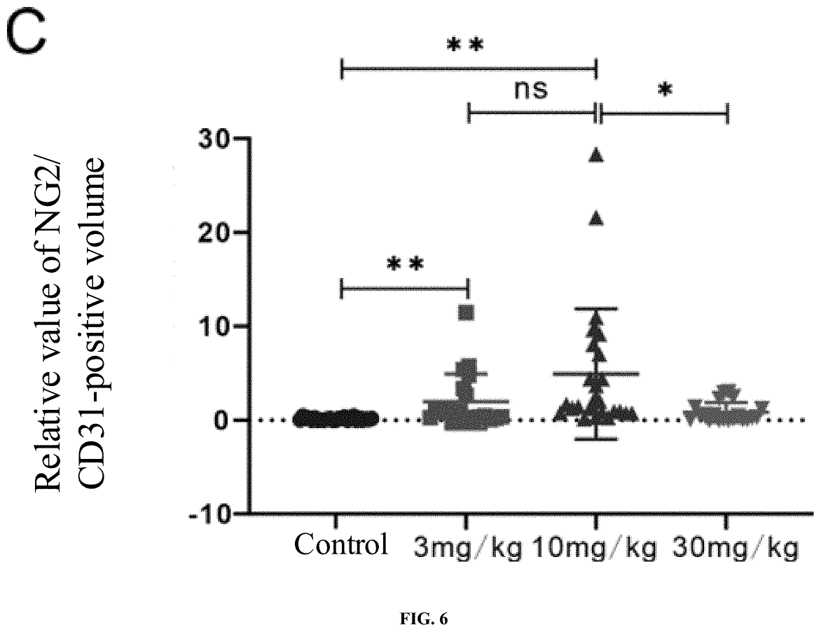

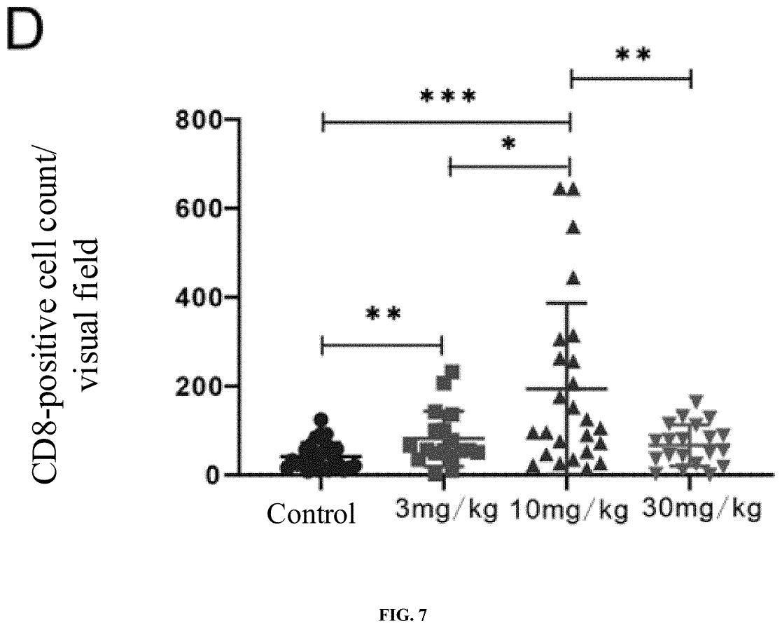

Immunohistochemistry results are shown in FIGS. 3, 4, 5, 6, and 7. All of the three dose gradients of lenvatinib may significantly reduce the microvessel density (CD31) in hepatoma tissue. However, only the 10 mg/kg/day dose group may significantly improve vascular normalization in tumor tissue, manifesting as the upregulation of the expression of peripheral cell marker molecule NG2; meanwhile, infiltrating CD8-positive T cells in the tumor tissue are also significantly increased, indicating that 10 mg/kg/day lenvatinib is a low dose suitable for improving vascular normalization of tumors and promoting the T cell infiltration.

### Example 2

Effects of administration of different doses of lenvatinib plus anti-PD-1 monoclonal antibody on the size of hepatoma xenograft.

1. Experimental Materials

(1) Drugs:

1) lenvatinib, chemical formula: C₂₁H₁₉ClN₄O₄, CAS NO: 417716-92-8;

2) anti-PD-1 monoclonal antibody: anti-mouse PD-1 (CD279);

(2) Cancer cells: mouse hepatoma cells (Hepa1-6); and

(3) Commercially available immunocompetent C57B/L mice.

2. Experimental Grouping

(1) Control group: blank control, namely hepatoma-bearing mice untreated with any drug;

(2) Low-dose lenvatinib group: hepatoma-bearing mice treated with low-dose lenvatinib;

(3) High-dose lenvatinib group: hepatoma-bearing mice treated with high-dose lenvatinib;

(4) Anti-PD-1 monoclonal antibody alone group: hepatoma-bearing mice treated with anti-PD-1 monoclonal antibody;

(5) Low-dose lenvatinib+anti-PD-1 monoclonal antibody group: hepatoma-bearing mice treated with low-dose lenvatinib and anti-PD-1 monoclonal antibody; and

(6) High-dose lenvatinib+anti-PD-1 monoclonal antibody group: hepatoma-bearing mice treated with high-dose lenvatinib and anti-PD-1 monoclonal antibody.

3. Detection of the effects of different doses of lenvatinib plus anti-PD-1 monoclonal antibody on hepatoma xenograft by subcutaneous tumorigenicity assay of immunocompetent C57B/L mice.

(1) Separately, 1×10⁶ mouse hepatoma cells (Hepa1-6) were implanted into the subcutaneous stratum of the armpits of 24 C57B/L mice aged 3-4 weeks. The mice were randomized into six groups: The mice were randomized into six groups: a blank control group, an anti-PD-1 monoclonal antibody alone group, a low-dose lenvatinib group, a high-dose lenvatinib group, a low-dose lenvatinib+anti-PD-1 monoclonal antibody group, and a high-dose lenvatinib+anti-PD-1 monoclonal antibody group.

(2) When the size of the subcutaneous tumor reached 100-200 mm³: each mouse of the anti-PD-1 monoclonal antibody alone group was intraperitoneally administered with 200 μg of anti-PD-1 monoclonal antibody every three days; each mouse of the low-dose lenvatinib group was orally administered with 10 mg/kg/day lenvatinib by gastric gavage once a day; each mouse of the high-dose lenvatinib group was orally administered with 30 mg/kg/day lenvatinib by gastric gavage once a day; each mouse of the low-dose lenvatinib group+anti-PD-1 monoclonal antibody was orally administered with 10 mg/kg/day lenvatinib by gastric gavage once a day and intraperitoneally administered with 200 μg of anti-PD-1 monoclonal antibody every three days; and each mouse of the high-dose lenvatinib group+anti-PD-1 monoclonal antibody was orally administered with 30 mg/kg/day lenvatinib by gastric gavage once a day and intraperitoneally administered with 200 μg of anti-PD-1 monoclonal antibody every three days. According to the existing literature, 30 mg/kg was a routine dose of lenvatinib (Selleck) for laboratory mice.

(3) The tumor size was measured every two days, and the difference in tumor size was compared among groups.

(4) After 3 weeks, the mice were sacrificed, and data statistics and reduction were conducted.

4. Experimental Results

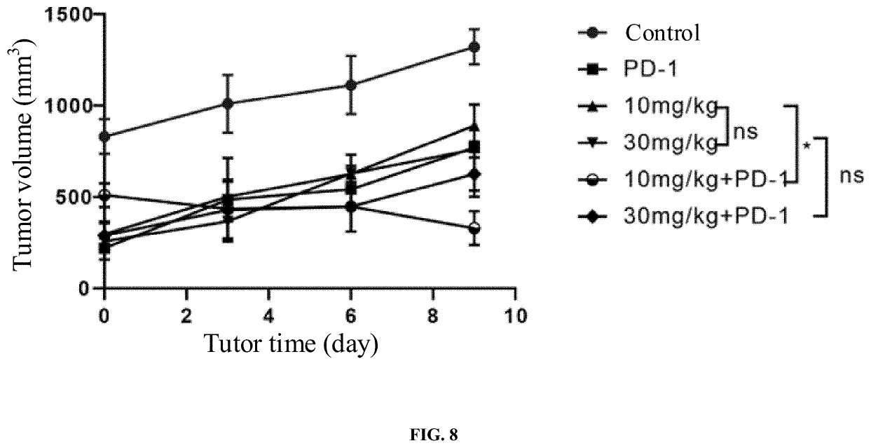

The results are shown in FIG. 8. Both low-dose lenvatinib (10 mg/kg/day) and high-dose lenvatinib (30 mg/kg/day) may inhibit the growth of hepatoma xenograft. There is no significant difference in tumor growth between low-dose and high-dose groups. However, tumor growth is significantly inhibited after administration of low-dose lenvatinib plus anti-PD-1 monoclonal antibody, while the inhibitory effect of administration of high-dose lenvatinib plus anti-PD-1 monoclonal antibody on tumor growth is not significantly enhanced.

Various embodiments of the disclosure may have one or more of the following effects. In some embodiments, the present disclosure may provide a new drug combination regimen for treatment of hepatoma, and the present disclosure may have an excellent application prospect in the aspect of the prophylaxis and treatment of the hepatoma.

Compared with the prior art, the present disclosure may have the following beneficial effects.

A thesis titled “Analysis of the Safety and Effectiveness of Lenvatinib Combined with PD-1 Inhibitor Compared with Sorafenib in the First-line Treatment of Advanced Hepatocellular Carcinoma” from Nanchang University further disclosed that administration of lenvatinib plus PD-1 could treat hepatoma. However, in this thesis, the doses of lenvatinib used were 8 mg/day (body weight <60 kg) and 12 mg/day (body weight >60 kg). These doses were converted into a dose used in mice (62.4 mg/kg/day) according to the equivalent dose conversion method (Table 1) in the Methodology of Pharmacological Experiments edited by Prof. Xu Shuyun. However, the dose used in the present disclosure is 10 mg/kg/day, which is significantly lower than the dose of lenvatinib disclosed so far. Therefore, in the present disclosure, the synergistic effect of 10 mg/kg/day lenvatinib on PD-1 is not obvious, which has outstanding advantages compared with the dose for treating hepatoma with lenvatinib plus PD-1 disclosed so far.

TABLE 1
The ratios of equivalent doses of human and animals converted by body surface area.

| | Mouse | Rat | Guinea pig | Rabbit | Cat | Monkey | Dog | Human |
|---|---|---|---|---|---|---|---|---|
| | (20 g) | (200 g) | (400 g) | (1.5 kg) | (2.0 kg) | (4.0 kg) | (12 kg) | (70 kg) |
| Mouse | 1.0 | 7.0 | 12.25 | 27.8 | 29.7 | 64.1 | 124.2 | 387.9 |
| Rat | 0.14 | 1.0 | 1.74 | 3.9 | 4.2 | 9.2 | 17.8 | 56.0 |
| Guinea pig | 0.08 | 0.57 | 1.0 | 2.25 | 2.4 | 5.2 | 10.2 | 31.5 |
| Rabbit | 0.04 | 0.25 | 0.44 | 1.0 | 1.08 | 2.4 | 4.5 | 14.2 |
| Cat | 0.03 | 0.23 | 0.41 | 0.92 | 1.0 | 2.2 | 4.1 | 13.0 |
| Monkey | 0.016 | 0.11 | 0.19 | 0.42 | 0.45 | 1.0 | 1.9 | 6.1 |
| Dog | 0.008 | 0.06 | 0.10 | 0.22 | 0.23 | 0.52 | 1.0 | 3.1 |
| Human | 0.0026 | 0.0018 | 0.031 | 0.07 | 0.018 | 0.06 | 0.32 | 1.0 |

In addition, the present disclosure finds that low-dose lenvatinib may improve the vascular “normalization” of hepatoma, which may promote the infiltration of T cells into the tumor tissue. Administration of low-dose lenvatinib plus anti-PD-1 monoclonal antibody has a significant synergistic effect on hepatoma. This mechanism is significantly different from all previous mechanisms underlying the treatment of hepatoma with lenvatinib plus PD-1. Therefore, such administration may be used for the prophylaxis and treatment of the hepatoma. The present disclosure may provide a new medication regimen for the treatment of hepatoma and may have an excellent application prospect in the aspect of the prophylaxis and treatment of the hepatoma.

The above descriptions are merely preferred implementations of the present disclosure. It should be noted that a person of ordinary skill in the art may further make several improvements and modifications without departing from the principle of the present disclosure, but such improvements and modifications should be deemed as falling within the protection scope of the present disclosure.

Many different arrangements of the various components depicted, as well as components not shown, are possible without departing from the spirit and scope of the present disclosure. Embodiments of the present disclosure have been described with the intent to be illustrative rather than restrictive. Alternative embodiments will become apparent to those skilled in the art that do not depart from its scope. A skilled artisan may develop alternative means of implementing the aforementioned improvements without departing from the scope of the present disclosure.

It will be understood that certain features and subcombinations are of utility and may be employed without reference to other features and subcombinations and are contemplated within the scope of the claims. Unless indicated otherwise, not all steps listed in the various figures need be carried out in the specific order described.

The disclosure claimed is:

1. An anti-hepatoma drug, comprising Lenvatinib and anti-PD-1 monoclonal antibody.

2. The anti-hepatoma drug of claim 1, wherein a dose of the lenvatinib is 10 mg/kg body weight/day.

3. The anti-hepatoma drug of claim 1, wherein a dose of the anti-PD-1 monoclonal antibody is 200 μg/kg body weight/3 days.

4. The anti-hepatoma drug of claim 1, further comprising pharmaceutically acceptable excipients or carriers.

5. A method for treating patients with hepatoma, wherein the patients were administered with the anti-hepatoma drug of claim 1.

6. The method of claim 5, wherein:
a dose of lenvatinib in the anti-hepatoma drug is 10 mg/kg body weight/day; and
a dose of anti-PD-1 monoclonal antibody is 200 μg/kg body weight/3 days.

7. The method of claim 5, wherein the anti-hepatoma drug is a medicament for inhibiting growth of hepatoma.

8. The method of claim 5, wherein the hepatoma is primary hepatocellular carcinoma.
